(19) United States
(12) Patent Application Publication
(10) Pub. No.: US 2023/0301180 A1
(43) Pub. Date: Sep. 21, 2023

(54) ORGANIC MATERIAL FOR ORGANIC ELECTRIC ELEMENT, METHOD FOR PRODUCING ORGANIC MATERIAL FOR ORGANIC ELECTRIC ELEMENT, AND ORGANIC ELECTRIC ELEMENT USING SAME

(71) Applicants: DUK SAN NEOLUX CO., LTD., Cheonan-si Chungcheongnam-do (KR); SAMSUNG DISPLAY CO., LTD., Yongin-si Gyeonggi-do (KR)

(72) Inventors: Dae Ho SONG, Cheonan-si (KR); Jong Gwang PARK, Cheonan-si (KR); Bum Sung LEE, Cheonan-si (KR); Jung Hwan PARK, Cheonan-si (KR); Soung Yun MUN, Cheonan-si (KR); Yun Suk LEE, Cheonan-si (KR); Sun Pil HWANG, Cheonan-si (KR); Joon Seok OH, Asan-si (KR)

(21) Appl. No.: 18/014,817

(22) PCT Filed: Jul. 5, 2021
(86) PCT No.: PCT/KR2021/008510

§ 371 Date: Jan. 6, 2023

(30) Foreign Application Priority Data
Jul. 13, 2020 (KR) 10-2020-0086374

(51) Int. Cl. H10K 85/60; H10K 50/11; H10K 71/00; C07D 209/86; C09K 11/06; C07D 409/12; C07D 495/04; C07D 403/04; C07D 405/14; C07D 403/10; C07D 251/24; C07D 401/10

(52) U.S. Cl. CPC H10K 85/636; H10K 50/11; H10K 71/311; C07D 209/86; C09K 11/06; H10K 85/633; C07D 409/12; C07D 495/04; H10K 85/6572; H10K 85/657; C07D 403/04; H10K 85/654; H10K 85/615; C07D 405/14; H10K 85/6574; C07D 403/10; H10K 85/626; C07D 251/24; C07D 401/10; C09K 2211/1018; H10K 85/6576

(57) ABSTRACT

Embodiments of the present invention relate to: an organic material for an organic electric element, which can improve the driving voltage, luminous efficiency, and service life characteristics of the organic electric element; a method for producing the organic material for an organic electric element; and an organic electric element using same.

### TECHNICAL FIELD

Embodiments of the present invention relate to an organic material for an organic electric element, a method for producing an organic material for an organic electric element, and an organic electric element using the same.

### BACKGROUND ART

Large-scale displays in the current portable display market requires more power than that consumed in conventional portable displays. Accordingly, power consumption becomes a critical factor for portable displays having a limited power source, such as a battery, and lifespan and efficiency issues should be addressed.

Such displays primarily include organic electric elements.

An organic electric element using the organic light emitting phenomenon typically has a structure including an anode and a cathode and an organic material layer therebetween. Here, the organic material layer often has a multi-layered structure composed of different materials to increase the efficiency and stability of the organic electric element, e.g., a hole injection layer, a hole transport layer, a light emitting layer, an electron transport layer and an electron injection layer.

Organic material layers may be deposited through a variety of processes. The power consumption, efficiency, and lifespan of the organic electric element may vary depending on the conditions of the deposition process, e.g., the organic material used in the deposition process.

### DETAILED DESCRIPTION OF THE INVENTION

### Technical Problem

Embodiments of the present invention may provide an organic material for an organic electric element, a method for producing an organic material for an organic electric element, and an organic electric element using the same, which may enhance the driving voltage, luminous efficiency and lifespan characteristics of the organic electric element.

### Technical Solution

In an aspect, embodiments of the present invention may provide a method for preparing an organic material for an organic electric element, which comprises a first step of preparing a first material including a first raw material and a second raw material, a second step of obtaining a second material by pulverizing the first material, and a third step of selecting a granular organic material a partial area or entire area of a surface of which has a needle shape from the second material and an organic electric element using the same.

In another aspect, embodiments of the present invention may provide an organic material for an organic electric element, comprising at least one kind of raw material, wherein a partial area or entire area of a surface of the organic material has a needle shape, and wherein the organic material has a granular form, and an organic electric element using the same.

### Advantageous Effects

According to the embodiments of the present invention, there may be provided an organic material for an organic electric element, a method for producing an organic material for an organic electric element, and an organic electric element using the same, which may achieve a reduced driving voltage, high luminous efficiency and long lifespan of the organic electric element by forming the organic electric element using an organic material.

### BRIEF DESCRIPTION OF THE DRAWINGS

FIG. 1 is a flowchart illustrating a method for producing an organic material according to an embodiment of the present invention;

FIG. 2 is a view illustrating a step of selecting an organic material according to an embodiment of the present invention;

FIG. 3 is a view illustrating a mixture according to an embodiment of the present invention;

FIG. 4 is an example view illustrating an organic light emitting element according to an embodiment of the present invention;

FIG. 5 is a graph illustrating the degree of gas generation depending on changes in pressure and temperature of an organic material according to an embodiment;

FIG. 6 is a graph illustrating the degree of gas generation depending on changes in pressure and temperature of an organic material according to a comparative example of the present invention;

FIG. 7 is a graph illustrating a result of qualitative analysis on the gas generated from an organic material according to a comparative example and an embodiment;

FIG. 8 is an image of a surface of an organic material according to an embodiment of the present invention; and

FIG. 9 is an image of a surface of an organic material according to a comparative example.

### MODE FOR CARRYING OUT THE INVENTION

Hereinafter, some embodiments of the present invention are described in detail with reference to the accompanying drawings. The same or substantially the same reference denotations are used to refer to the same or substantially the same elements throughout the specification and the drawings. When determined to make the subject matter of the present invention unclear, the detailed of the known art or functions may be skipped. The terms “comprises” and/or “comprising,” “has” and/or “having,” or “includes” and/or “including” when used in this specification specify the presence of stated features, regions, integers, steps, operations, elements, and/or components, but do not preclude the presence or addition of one or more other features, regions, integers, steps, operations, elements, components, and/or groups thereof. As used herein, the singular forms “a”, “an” and “the” are intended to include the plural forms as well, unless the context clearly indicates otherwise.

Such denotations as “first,” “second,” “A,” “B,” “(a),” and “(b),” may be used in describing the components of the present invention. These denotations are provided merely to distinguish a component from another, and the essence of the components is not limited by the denotations in light of order or sequence.

In describing the positional relationship between components, when two or more components are described as “connected”, “coupled” or “linked”, the two or more components may be directly “connected”, “coupled” or “linked”, or another component may intervene. Here, the other component may be included in one or more of the two or more components that are “connected”, “coupled” or “linked” to each other.

In relation to components, operational methods or manufacturing methods, when A is referred to as being “after,” “subsequent to,” “next,” and “before,” A and B may be discontinuous from each other unless mentioned with the term “immediately” or “directly.”

When a component is designated with a value or its corresponding information, the value or the corresponding information may be interpreted as including a tolerance that may arise due to various factors (e.g., process factors, internal or external impacts, or noise).

When an embodiment may be implemented otherwise, the specific processing order may be different from the described order. For example, two processes described as successive may be performed substantially simultaneously or in the opposite order.

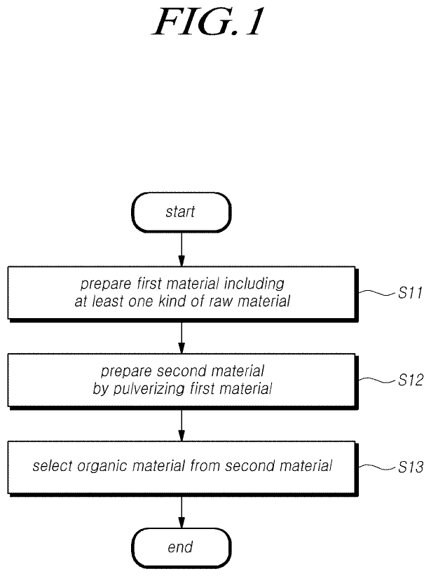

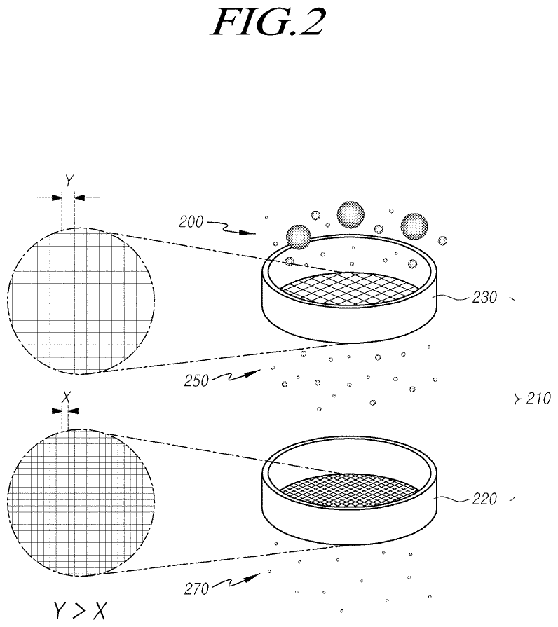

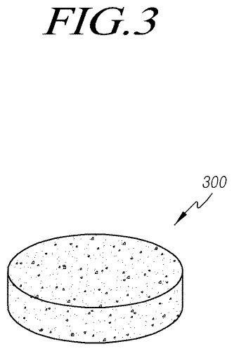

FIG. 1 is a flowchart illustrating a method for producing an organic material according to an embodiment of the present invention. FIG. 2 is a view illustrating a step of selecting an organic material according to an embodiment of the present invention. FIG. 3 is a view illustrating a formed state of an organic material according to an embodiment of the present invention.

Referring to FIG. 1, a method for producing an organic material according to an embodiment of the present invention includes a first step of preparing a first material including at least one kind of raw materials. (S11)

The first material may be prepared using one kind of raw material.

The raw material used to prepare the first material may include at least one kind of organic material, but the present invention is not limited thereto. Here, the raw material used to prepare the first material may be in the form of a powder (or may be referred to as a powder form).

In the present invention, the raw material may be a material containing at least one species of compound among compounds containing an amino group, compounds containing azines, and compounds containing a polycyclic ring, but the present invention is not limited thereto.

The first material of the present invention may include two or more raw materials.

For example, the first material may be prepared of a first raw material and a second raw material.

At least one of the first raw material and the second raw material included in the first material may include at least one kind of organic material, but the present invention is not limited thereto.

In this case, the first step may include melting each of the first raw material and the second raw material, and then physically mixing the first raw material and the second raw material which are solidified.

However, the present invention is not limited thereto, and as another example, the first material may include a third raw material and a fourth raw material.

In this case, the first step may include physically mixing the third raw material and the fourth raw material, and then melting the mixed third and fourth raw materials. Here, at least one of the third raw material and the fourth raw material may include at least one kind of organic material, but the present invention is not limited thereto.

Each raw material may be mixed in the atmosphere or be mixed with moisture blocked. In this case, the first raw material and the second raw material may be mixed in a weight ratio of 1:1 to 1:9 or 1:1 to 9:1, preferably, in a 1:1 ratio. Further, the third raw material and the fourth raw material may also be mixed in a weight ratio of 1:1 to 1:9 or 1:1 to 9:1, but the present invention is not limited thereto, and the relative weight ratio of the raw materials may vary.

When each raw material is melted in an environment exposed to moisture and oxygen, impurities may be included. Accordingly, the melting process may be performed in a vacuum state, but the melting process of the present invention is not limited thereto.

The melting process of the first step may include a step of temperature-treating each raw material.

The melting process may include heat-treating each of the first raw material and the second raw material, or physically mixing the first raw material and the second raw material, and then heat-treating the mixed materials.

The temperature during the heat treatment step of the melting process may be selected from among 50° C. to 70° C. lower temperatures (hereinafter, referred to as heat-treatment temperature) than the temperature at which the weight reduction of the raw material occurs by 0.5% upon measuring the pyrolysis temperature (Td) of the raw material.

Specifically, when the first step is performed by melting each of the first raw material and the second raw material, solidifying the first raw material and the second raw material, and then physically mixing the first raw material and the second raw material, the first raw material and the second raw material may be thermally treated at different heat-treatment temperatures.

Further, when the first step is performed in the order of physically mixing the third raw material and the fourth raw material and then melting the mixed third and fourth raw materials and then solidifying them, the heat treatment process may be performed at the higher heat treatment temperature of the heat treatment temperature of the third raw material and the heat treatment temperature of the fourth raw material. In other words, when two or more kinds of raw materials are used to form the first material, and the two or more kinds of raw materials are physically mixed and then simultaneously thermally treated, the higher heat treatment temperature of the respective heat treatment temperatures of the raw materials may be selected, and the heat treatment step of the melting process may be performed.

Further, in the temperature treatment step, the pressure may be selected in a range of 10⁻⁶ to 10⁻³ Torr.

In the heat treatment step, when heat is applied to the raw materials, the whole or part of the raw material may go through a liquid state to generate an impurity gas.

As described above, the raw material that has undergone the heat treatment step may be solidified at a temperature lower than the temperature of the heat treatment step. For example, the raw material that has undergone the melting process may be solidified at room temperature, but the present invention is not limited thereto.

Thereafter, the first material solidified through the melting process is pulverized to prepare a second material. (S12) Next, an organic material is selected from the pulverized second material. (S13)

The selected organic material may be in the form of granules (grains) in which the shape of a partial area or the entire area of the surface is a needle shape.

The process of selecting the organic material is discussed below in detail with reference to FIG. 2.

Referring to FIG. 2, the pulverized second material 200 may be separated into the organic material 250 and the residue 270 through a separator 210.

The separator 210 may include a first filter 220 and a second filter 230 disposed on the first filter 220 and spaced apart from the first filter 220. Here, the particle diameter X of the first filter 220 may be smaller than the particle diameter Y of the second filter 230. For example, the particle diameter X of the first filter 220 may be 0.1 mm, and the particle diameter Y of the second filter 230 may be 0.5 mm or less. First, the pulverized second material 200 may be passed through the second filter 230 of the separator 210. For example, when the particle diameter Y of the second filter 230 is 0.5 mm, only particles having a particle diameter of 0.5 mm or less in the pulverized second material 200 may pass through the second filter 230. Particles having a particle diameter exceeding 0.5 mm in the pulverized second material 200 do not pass through the second filter 230 but remain on the second filter 230.

Particles having a particle size of 0.1 mm or less among the particles passing through the second filter 230 may pass through the first filter 220. Particles that do not pass through the first filter 220 remain on the first filter 220.

The particles remaining on the first filter 220 may be particles corresponding to the organic material 250. The particles that have passed through the first filter 220 may be the residue 270.

The size of the particles constituting the organic material 250 may exceed 0.1 mm and be 0.5 mm or less. The size of the residue 270 may be 0.1 mm or less.

The organic material 250 including particles having a size exceeding 0.1 mm and being 0.5 mm or less may be formed into a specific shape.

For example, as illustrated in FIG. 3, the organic material 250 may be compression-formed into a formed body 300 shaped as a disk or a polygon, but the present invention is not limited thereto.

The formed body 300 of the organic material 250 may be used in a process of forming an organic electric element.

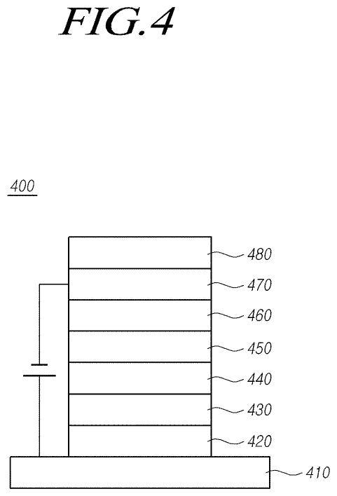

The structure of the organic electric element according to an embodiment of the present invention is discussed below with reference to FIG. 4.

FIG. 4 is an example view illustrating an organic light emitting element according to an embodiment of the present invention.

An organic electric element 400 according to an embodiment of the present invention may include a first electrode 410, a second electrode 470, and an organic material layer including a compound according to the present invention between the first electrode 410 and the second electrode 470, formed on a substrate, and may further include, or may not include, a capping layer 480.

The first electrode 410 of FIG. 1 may be an anode, and the second electrode 470 may be a cathode. In the inverted type, the first electrode may be a cathode, and the second electrode may be an anode.

The organic material layer may include a hole injection layer 420, a hole transport layer 430, a light emitting layer 440, an electron transport layer 450, and an electron injection layer 460. Specifically, the hole injection layer 420, the hole transport layer 430, the light emitting layer 440, the electron transport layer 450, and the electron injection layer 460 may be sequentially disposed on the first electrode 410.

Meanwhile, although not shown in FIG. 1, a light emitting auxiliary layer may be further disposed between the hole transport layer 430 and the light emitting layer 440. An electron transport auxiliary layer or a buffer layer may be further disposed between the light emitting layer 440 and the electron transport layer 450.

The formed body 300 of the organic material 250 of the present invention may be used as a material forming the hole injection layer 420, the hole transport layer 430, the light emitting layer 440, the electron transport layer 450 or the electron injection layer 460. For example, the formed body 300 of the organic material 250 of the present invention may be used as a host material of the light emitting layer 440.

The organic electric element 400 according to embodiments of the present invention may be prepared by various deposition methods. The organic electric element 400 may be prepared using a deposition method, such as physical vapor deposition (PVD) or chemical vapor deposition (CVD), e.g., by depositing a metal or a metal oxide having conductivity or an alloy thereof on a substrate to form the anode 410, forming an organic material layer including a hole injection layer 420, a hole transport layer 430, a light emitting layer 440, an electron transport layer 450 and an electron injection layer 460 thereon, and then depositing a material which may be used as the cathode 470 thereon.

As such, in preparing the organic electric element 400, the deposition process is an essential process.

During the deposition process, an organic material for an organic electric element is deposited on a substrate by applying a specific temperature under a specific pressure. At this time, a gas may be generated depending on the shape of the organic material for the organic electric element. The gas may contaminate the inside of the deposition machine (e.g., the inside of the chamber), adversely affecting the organic electric element and shortening the life of the deposition machine.

As described above, the method for preparing the organic material 250 for an organic electric element of the present invention includes a step of obtaining a second material through a melting process of the first material. After the melting process, a process of pulverizing the solidified second material is performed.

The pulverized second material may be divided into a powder form and a granular form depending on the size of the particles. The organic material that has undergone the organic material preparing method of the present invention has a granular form. A compound for an organic electric element formed of an organic material containing only particles in the granular form may offset gas emissions, preventing the performance degradation of the organic electric element and the deposition machine.

Here, the granular particles mean particles that have a particle diameter exceeding 0.1 mm and a size of 0.5 mm or less.

The organic material 250 for an organic electric element of the present invention may be prepared through the organic material including particles in the granular form having a particle diameter exceeding 0.1 mm and a size of 0.5 mm or less.

However, the organic material for the organic electric element formed of powder-type particles (composition corresponding to the residue) having a smaller particle diameter than the granular-type particles has a larger volume relative to the same mass than the organic material 250 for the organic electric element of the present invention prepared with granular particles. This means that the density of the organic material prepared in the form of powder particles is lower than the density of the organic material 250 for the organic electric element of the present invention. The low-density organic material has a larger surface area exposed to air than the high-density organic material 250 of the present invention.

An increase in the surface area of the organic material means an increase in the area bonded with impurities, and as the amount of impurities bonded to the surface of the organic material increases, the amount of gas generated from the organic material in the deposition process increases.

In other words, since the organic material prepared in powder form contains more impurities than the organic material 250 for the organic electric element of the present invention, it contaminates the inside of the deposition machine during the deposition process of the organic electric element and degrades the performance of the organic electric element and deposition machine.

Comparison in the amount of gas emissions between the organic material prepared in powdered particles (hereinafter, referred to as organic material according to the comparative example) and the organic material for the organic electric element of the present invention (hereinafter, referred to as organic material according to the embodiment) is made below.

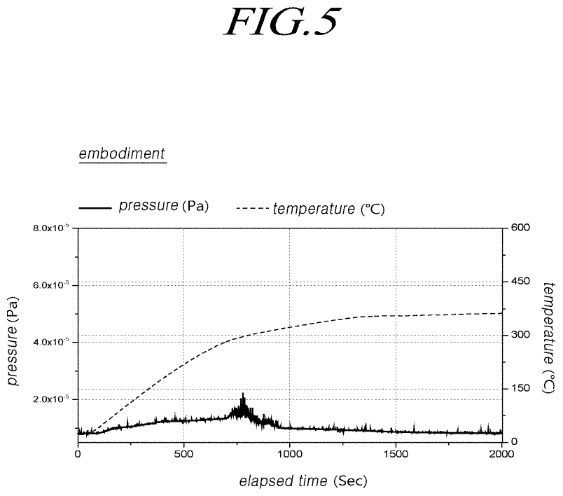

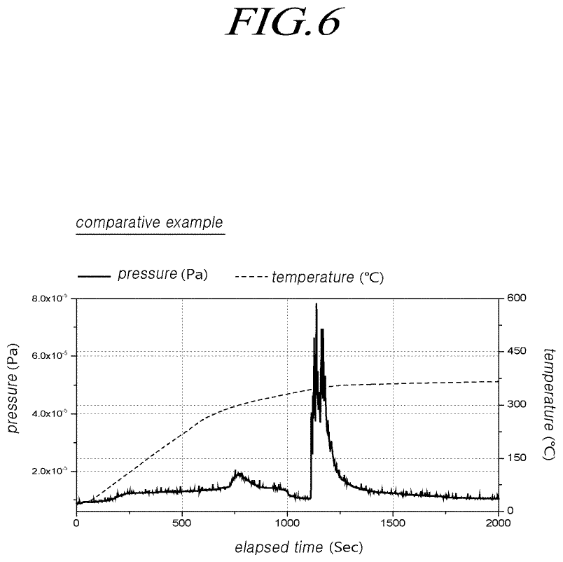

FIG. 5 is a graph illustrating the degree of gas generation depending on changes in pressure and temperature of an organic material according to an embodiment. FIG. 6 is a graph illustrating the degree of gas generation depending on changes in pressure and temperature of an organic material according to the comparative example.

In FIGS. 5 and 6, the x-axis denotes the elapsed time, and the y-axis denotes the pressure (solid line) and temperature (dashed line).

Referring to FIG. 5, in the case of the organic material according to the embodiment, it may be seen that a slight pressure change occurs when 750 seconds have elapsed. In the experiment, the temperature was set to converge to 375° C. over time. The converged temperature is an arbitrary temperature set to identify whether gas is generated according to the temperature, and the converged temperature may be changed depending on the type of the raw material.

It may be seen that the change in the proportion of the organic material according to the embodiment occurs at the time of a pressure change (e.g., elapsed time between 750 seconds and 900 seconds). In this case, it may be seen that a pressure change appears due to the gas generated from the organic material according to the embodiment. Here, the magnitude of the pressure change of the organic material according to the embodiment may correspond to the amount of the gas generated from the organic material according to the embodiment.

Referring to FIG. 6, it may be seen that in the case of the organic material according to the comparative example, a large pressure change occurs when 1100 seconds have elapsed. Further, like in the embodiment, in the experiment, the temperature was set to converge to 375° C. over time. The converged temperature is an arbitrary temperature set to identify whether gas is generated according to the temperature, and the converged temperature may be changed depending on the type of the raw material.

It may be seen that the change in the proportion of the organic material according to the comparative example occurs primarily at the time of a pressure change (e.g., elapsed time between 1100 seconds and 1300 seconds). Here, the magnitude of the pressure change of the organic material according to the comparative example may correspond to the amount of the gas generated from the organic material according to the comparative example.

The time when the pressure change of the organic material occurs according to the embodiment and the comparative example shown in FIGS. 5 and 6 may be changed depending on, e.g., the amount and type of the organic material used in the experiment.

In FIGS. 5 and 6, the amount of the organic material according to the embodiment and the amount of the organic material according to the comparative example as used in the experiment were the same, and it may be seen that the magnitude of the pressure changed by applying heat to the organic material according to the embodiment is smaller than the magnitude of the pressure changed by applying heat to the organic material according to the comparative example.

In other words, it may be seen that the amount of the gas generated from the organic material according to the embodiment is significantly smaller than the amount of the gas generated from the organic material according to the comparative example. It may be seen that the organic material according to the comparative example, which includes powder having a smaller size than the granules of the material included in the organic material according to the embodiment, emits more gases than the organic material according to the embodiment at a higher temperature than room temperature.

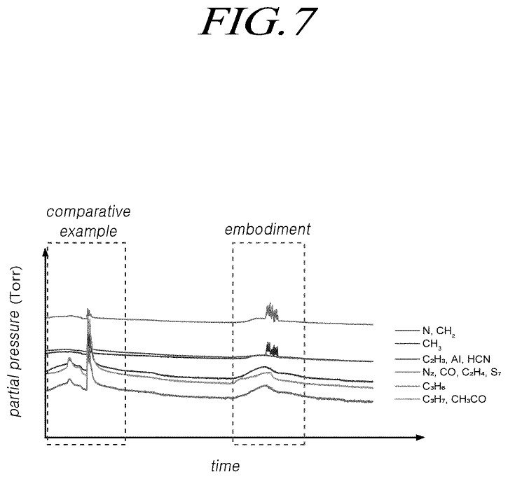

FIG. 7 is a graph illustrating a result of qualitative analysis on the gas generated from an organic material according to a comparative example and an embodiment.

The type of the gas generated from the organic materials according to the comparative example and the embodiment may be predicted through a residual gas analyzer (RGA).

In FIG. 7, the x-axis means the time, and the y-axis means the partial pressure.

Referring to FIG. 7, it may be seen that gases (gases due to the impurities included in the organic material according to the comparative example) including N, CH₂, CH₃, C₂H₃, Al, HCN, N₂, CO, C₂H₄, Si, C₃H₆, C₃H₇ and CH₃CO were generated from the organic material according to the comparative example. On the other hand, it may be seen that gases, such as N, CH₂ and CH₃ (gases due to the impurities included in the organic material according to the embodiment) were generated from the organic material according to the embodiment. In other words, it may be seen that the amount of impurities included in the organic material according to the comparative example is larger than the amount of impurities included in the organic material according to the embodiment.

CH₂, CH₃, C₂H₃, CO, C₂H₄, C₃H₆, C₃H₇ and CH₃CO of FIG. 7 may be empirical formulas (simply representing the ratio of each element). N and N₂ may be gases generated from the organic material or atmospheric gases used in the analysis process using the residual gas analyzer.

Accordingly, as illustrated in FIG. 7, it may be seen that the types of gases generated from the organic material according to the comparative example are more than the types of gases generated from the organic material according to the embodiment and that the pressure variation due to the gases generated from the organic material according to the comparative example is larger than the pressure variation due to the gases generated from the organic material according to the embodiment.

Referring to FIGS. 5 to 7, it may be seen that even for the same compounds, the difference in the content of the gas generated at the same temperature and the same pressure is large. Since the amount and type of gases generated from the organic material according to the embodiment are significantly smaller than the amount and type of gases generated from the organic material according to the comparative example, if the organic electric element is prepared using the organic material according to the embodiment, it is possible to suppress property degradation due to the organic material according to the embodiment.

In contrast, if a high temperature is applied at room temperature, the organic material according to the comparative example generates much gas, and the generated gas includes CH₂, CH₃, C₂H₃, Al, HCN, CO, C₂H₄, Si, C₃H₆, C₃H₇ and CH₃CO, which may affect the properties of the organic electric element.

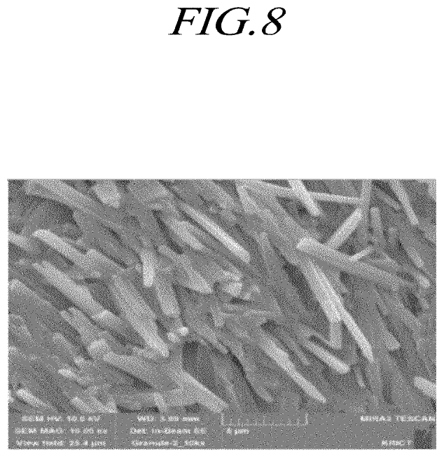

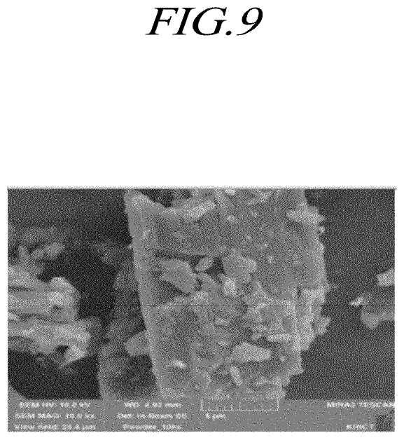

Subsequently, comparison in surface characteristics between the organic material according to the embodiment and the organic material according to the comparative example is described with reference to FIGS. 8 and 9.

FIG. 8 is a surface image of an organic material according to the embodiment of the present invention, and FIG. 9 is a surface image of an organic material according to the comparative example.

FIGS. 8 and 9 are scanning electron microscope (SEM) images of the respective surfaces of the organic materials, as magnified 10,000 times.

Referring to FIG. 8, it may be seen that the surface of the organic material according to the embodiment has a needle-shaped surface structure.

On the other hand, referring to FIG. 9, it may be seen that the surface of the organic material according to the comparative example has an irregular shape.

In other words, although prepared of the same raw materials, the organic material according to the embodiment and the organic material according to the comparative example may have different surface shapes.

Comparison in characteristics between the organic electric element including the organic material according to the embodiment of the present invention and the organic electric element including the organic material according to the comparative example is made below.

Evaluation of Preparation of Organic Electric Element

### [Embodiment 1] Red Organic Light Emitting Element (Light Emitting Auxiliary Layer)

An organic electric element was prepared, according to a typical method, using the organic material of the present invention, obtained through the above-described method, as the light emitting auxiliary layer material. First, an N1-(naphthalen-2-yl)-N4, N4-bis(4-(naphthalen-2-yl(phenyl)amino)phenyl)-N1-phenylbenzene-1,4-diamine (abbreviated as 2-TNATA) film, as the hole injection layer, was vacuum-deposited, to a thickness of 60 nm, on an ITO layer (anode) formed on a glass substrate. Subsequently, N, N′-Bis(1-naphthalenyl)-N, N′-bis-phenyl-(1, 1′-Biphenyl)-4, 4′-diamine (hereinafter, abbreviated as NPB) was vacuum-deposited to a thickness of 60 nm, forming the hole transport layer. Subsequently, organic material 1 in the granular form according to an embodiment of the present invention, as the light emitting auxiliary layer material (hereinafter, referred to as organic material 1 according to the embodiment) was vacuum-deposited to a thickness of 20 nm to form the light emitting auxiliary layer material. After the light emitting auxiliary layer was formed, CBP[4,4′-N,N′-dicarbazole-biphenyl], as a host, was used on the light emitting auxiliary layer, and was doped with (piq)₂Ir(acac) [bis-(1-phenyl isoquinolyl)iridium(²ate], as a dopant, in a weight ratio of 95:5, depositing a 30 nm-thick light emitting layer on the light emitting auxiliary layer. (1,1′-bisphenyl)-4-oleato)bis(2-methyl-8-quinolineoleato)aluminum (hereinafter abbreviated as BAlq) was vacuum-deposited to a thickness of 10 nm, as a hole blocking layer, and a tris(8-quinolinol)aluminum (hereinafter abbreviated as Alq₃) film, as the electron transport layer, was formed to a thickness of 40 nm. Thereafter, LiF, which is an alkali metal halide, as an electron injection layer, was deposited to a thickness of 0.2 nm as an electron injection layer, and Al was then deposited to a thickness of 150 nm and used as a cathode, preparing an organic light emitting element.

[Organic Material 1 According to the Embodiment]

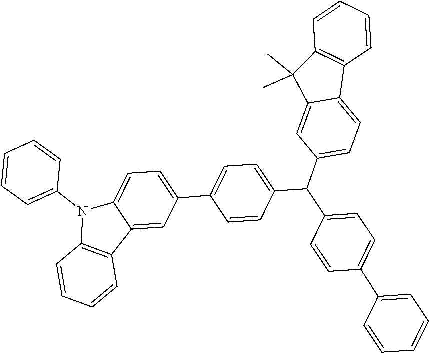 [SMILES: CC1(C)c2ccccc2-c2ccc(C(c3ccc(-c4ccccc4)cc3)c3ccc(-c4ccc5c(c4)c4ccccc4n5-c4ccccc4)cc3)cc21]

### Embodiment 2 to Embodiment 5

The organic electric element was prepared by the same method as that in embodiment 1 except that organic materials 2 to 5 according to embodiments of the present invention described below were used as the light emitting auxiliary layer material, instead of organic material 1 according to the embodiment of the present invention.

[Organic Material 2 According to the Embodiment] [Organic Material 3 According to the Embodiment]

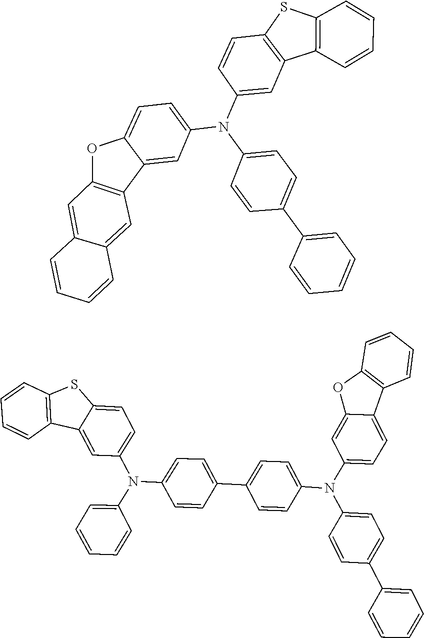 [SMILES: c1ccc(-c2ccc(N(c3ccc4oc5cc6ccccc6cc5c4c3)c3ccc4sc5ccccc5c4c3)cc2)cc1 ; c1ccc(-c2ccc(N(c3ccc(-c4ccc(N(c5ccccc5)c5ccc6sc7ccccc7c6c5)cc4)cc3)c3ccc4c(c3)oc3ccccc34)cc2)cc1]

[Organic Material 4 According to the Embodiment] [Organic Material 5 According to the Embodiment]

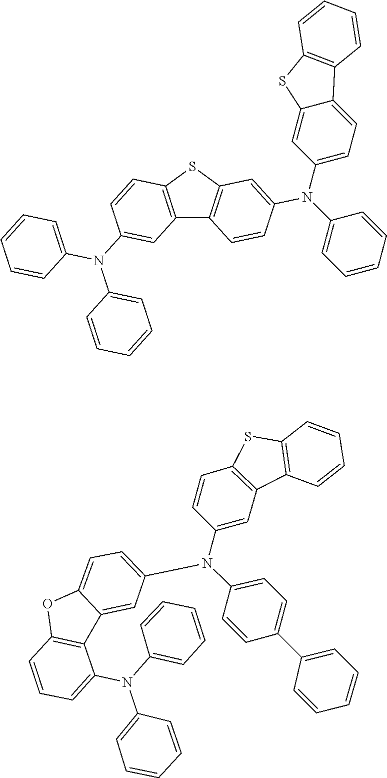 [SMILES: c1ccc(N(c2ccc3c(c2)sc2ccccc23)c2ccc3c(c2)sc2ccc(N(c4ccccc4)c4ccccc4)cc23)cc1 ; c1ccc(-c2ccc(N(c3ccc4sc5ccccc5c4c3)c3ccc4oc5cccc(N(c6ccccc6)c6ccccc6)c5c4c3)cc2)cc1]

### Comparative Examples 1 to 5

The organic electric element was prepared by the same method as that in embodiment 1 except that a powdered compound was used rather than the granular organic material, as the light emitting auxiliary layer material.

A forward bias DC voltage was applied to the organic electric elements prepared according to embodiments 1 to 5 of the present invention and comparative examples 1 to 5, and the electroluminescence (EL) characteristics were measured with PR-650 of Photo Research Inc., and the T95 lifespan was measured on the measurement result through a lifespan meter manufactured by Mcscience Inc. at the 2500 cd/m² reference luminance. The measurement result is as shown in Table 1 below. In Table 1, the numbers marked after the powders and granules of the organic materials are for distinguishing the types of powders and granules applied to the respective comparative examples and the embodiments.

TABLE 1

| | | Driving | | | | | | |
|---|---|---|---|---|---|---|---|---|
| | Organic | voltage | Current | Luminance | Efficiency | | CIE | |
| | material | (V) | (mA/cm2) | (cd/m2) | (cd/A) | T(95) | x | y |
| Comparative | Powder 1 | 5.3 | 11.5 | 2500 | 21.8 | 88.4 | 0.61 | 0.34 |
| example (1) | | | | | | | | |
| Comparative | Powder 2 | 5.0 | 10.7 | 2500 | 23.4 | 103.1 | 0.61 | 0.31 |
| example (2) | | | | | | | | |
| Comparative | Powder 3 | 4.9 | 11.6 | 2500 | 21.5 | 99.9 | 0.61 | 0.34 |
| example (3) | | | | | | | | |
| Comparative | Powder 4 | 4.8 | 11.3 | 2500 | 22.1 | 102.5 | 0.63 | 0.34 |
| example (4) | | | | | | | | |
| Comparative | Powder 5 | 5.0 | 11.2 | 2500 | 22.3 | 101.3 | 0.64 | 0.33 |
| example (5) | | | | | | | | |
| Embodiment (1) | Granule 1 | 5.2 | 11.2 | 2500 | 22.4 | 118.4 | 0.60 | 0.30 |
| Embodiment (2) | Granule 2 | 4.8 | 9.7 | 2500 | 25.7 | 137.3 | 0.64 | 0.33 |
| Embodiment (3) | Granule 3 | 4.8 | 10.5 | 2500 | 23.9 | 134.1 | 0.61 | 0.32 |
| Embodiment (4) | Granule 4 | 4.7 | 10.3 | 2500 | 24.2 | 135.7 | 0.64 | 0.33 |
| Embodiment (5) | Granule 5 | 4.8 | 10.1 | 2500 | 24.8 | 133.5 | 0.65 | 0.32 |

### [Embodiment 6] Red Organic Light Emitting Element (Phosphorescent Host)

An organic electric element was prepared, according to a typical method, using the organic material obtained through synthesis, as the light emitting host material. First, an N1-(naphthalen-2-yl)-N4, N4-bis(4-(naphthalen-2-yl(phenyl)amino)phenyl)-N1-phenylbenzene-1,4-diamine (abbreviated as 2-TNATA) film was vacuum-deposited on an ITO layer (anode) formed on a glass substrate to form a 60 nm-thick hole injection layer, and then, 4,4-bis[N-(1-naphthyl)-N-phenylamino]biphenyl (hereinafter abbreviated as -NPD), as the hole transport compound, was deposited to a thickness of 60 nm on the hole injection layer to form a hole transport layer. Organic material 6, as a host, was used on the hole transport layer, and was doped with (piq)₂Ir(acac) [bis-(1-phenylisoquinolyl)iridium(III)acetylacetonate], as a dopant material, in a weight ratio of 95:5, forming a 30 nm-thick light emitting layer. Subsequently, (1,1′-bisphenyl)-4-oleato)bis(2-methyl-8-quinolineoleato)aluminum (hereinafter abbreviated as BAlq) was vacuum-deposited to a thickness of 10 nm, as a hole blocking layer, and a tris(8-quinolinol)aluminum (hereinafter abbreviated as Alq₃) film, as the electron transport layer, was formed to a thickness of 40 nm. Thereafter, LiF, which is an alkali metal halide, as an electron injection layer, was deposited to a thickness of 0.2 nm as an electron injection layer, and Al was then deposited to a Thickness of 150 nm and Used as a Cathode, Preparing an Organic Electric Element.

[Organic material 6 according to the embodiment]

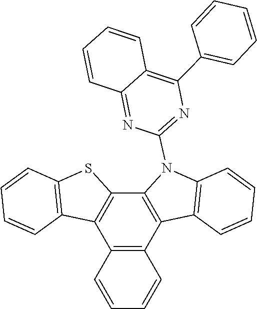 [SMILES: c1ccc(-c2nc(-n3c4ccccc4c4c5ccccc5c5c6ccccc6sc5c43)nc3ccccc23)cc1]

### Embodiment 7 to Embodiment 9

The organic electric element was prepared by the same method as that in embodiment 6 except that the compound of the present invention described below was used as the host material of the light emitting layer, instead of organic material 6 according to the embodiment of the present invention.

[Organic Material 7 According to the Embodiment] [Organic Material 8 According to the Embodiment]

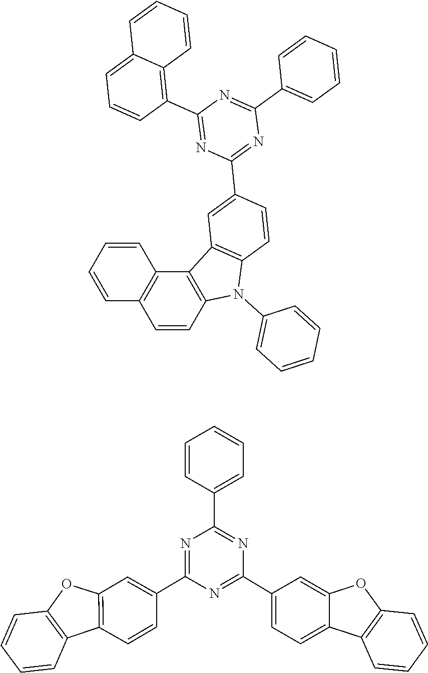 [SMILES: c1ccc(-c2nc(-c3ccc4c(c3)c3c5ccccc5ccc3n4-c3ccccc3)nc(-c3cccc4ccccc34)n2)cc1 ; c1ccc(-c2nc(-c3ccc4c(c3)oc3ccccc34)nc(-c3ccc4c(c3)oc3ccccc34)n2)cc1]

[Organic Material 9 According to the Embodiment]

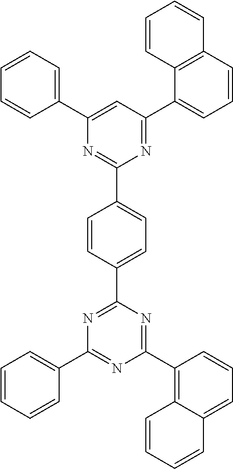 [SMILES: c1ccc(-c2cc(-c3cccc4ccccc34)nc(-c3ccc(-c4nc(-c5ccccc5)nc(-c5cccc6ccccc56)n4)cc3)n2)cc1]

### Embodiment 10 to Embodiment 17

The organic electric element was prepared by the same method as that in embodiment 6 by simply mixing (physically mixing) one of organic material 6 to organic material 9 according to the embodiment, as the host material of the light emitting layer, and another material (or heterogeneous compound) in a weight ratio of 5:5. The heterogeneous compound is as shown in Table 2.

### Embodiment 18 to Embodiment 25

An organic material was formed by mixing a raw material having the same structural formula as each of organic material 6 according to the embodiment of the present invention to organic material 9 according to the embodiment, as the host material of the light emitting layer, and a heterogeneous compound in a weight ratio of 5:5 and thermal-treating them (sublimation refining, physically mixing the materials and then melting them) to form an organic material, and then, the organic electric element was prepared by the same method as that in embodiment 6. The other material (or heterogeneous compound) is as shown in Table 2.

### Comparative Examples 6 to 25

The organic electric element was prepared by the same method as that in embodiment 6 except that a powdered organic material was used rather than the granular organic material, as the light emitting layer material.

### Comparative Examples 26 to 29

The organic electric element was prepared by the same method as that in embodiment 6 except that the state of the heterogeneous compound of the present invention as the light emitting layer material was applied to only one of a granular compound or a powdered compound and was used.

A forward bias DC voltage was applied to the organic electric elements prepared according to embodiments 6 to 26 and comparative examples 6 to 26, and the electroluminescence (EL) characteristics were measured with PR-650 of Photo Research Inc., and the T95 lifespan was measured on the measurement result through a lifespan meter manufactured by Mcscience Inc. at the 2500 cd/m² reference luminance. Table 2 below shows the results of element preparation and evaluation. In Table 2, the numbers marked after the powders and granules of the first and second materials are for distinguishing the types of powders and granules applied to the respective comparative examples and the embodiments.

TABLE 2

| | | | Driving | | | | | | |
|---|---|---|---|---|---|---|---|---|---|
| | First | Second | voltage | Current | Luminance | Efficiency | | CIE | |
| | material | material | (V) | (mA/cm2) | (cd/m2) | (cd/A) | T(95) | x | y |
| Comparative | Powder 6 | — | 5.5 | 11.2 | 2500.0 | 22.4 | 92.7 | 0.61 | 0.34 |
| example (6) | | | | | | | | | |
| Comparative | Powder 7 | — | 4.9 | 11.8 | 2500.0 | 21.1 | 91.3 | 0.64 | 0.30 |
| example (7) | | | | | | | | | |
| Comparative | Powder 8 | — | 4.8 | 12.2 | 2500.0 | 20.5 | 90.1 | 0.62 | 0.33 |
| example (8) | | | | | | | | | |
| Comparative | Powder 9 | — | 4.7 | 12.6 | 2500.0 | 19.8 | 80.4 | 0.62 | 0.33 |
| example (9) | | | | | | | | | |
| Comparative | Powder 6 | Powder 2 | 5.6 | 10.1 | 2500.0 | 24.8 | 95.4 | 0.60 | 0.33 |
| example (10) | | | | | | | | | |
| Comparative | Powder 7 | Powder 2 | 5.0 | 10.4 | 2500.0 | 24.1 | 92.1 | 0.64 | 0.33 |
| example (11) | | | | | | | | | |
| Comparative | Powder 8 | Powder 2 | 4.8 | 10.6 | 2500.0 | 23.5 | 90.8 | 0.65 | 0.31 |
| example (12) | | | | | | | | | |
| Comparative | Powder 9 | Powder 2 | 4.7 | 11.5 | 2500.0 | 21.8 | 85.9 | 0.65 | 0.34 |
| example (13) | | | | | | | | | |
| Comparative | Powder 6 | Powder 4 | 5.5 | 10.5 | 2500.0 | 23.7 | 94.2 | 0.63 | 0.30 |
| example (14) | | | | | | | | | |
| Comparative | Powder 7 | Powder 4 | 4.8 | 10.8 | 2500.0 | 23.2 | 91.6 | 0.64 | 0.32 |
| example (15) | | | | | | | | | |
| Comparative | Powder 8 | Powder 4 | 4.7 | 11.2 | 2500.0 | 22.4 | 90.2 | 0.65 | 0.31 |
| example (16) | | | | | | | | | |
| Comparative | Powder 9 | Powder 4 | 4.6 | 12.0 | 2500.0 | 20.9 | 83.7 | 0.60 | 0.32 |
| example (17) | | | | | | | | | |
| Comparative | Powder 6 | Powder 2 | 5.6 | 9.9 | 2500.0 | 25.2 | 95.5 | 0.61 | 0.33 |
| example (18) | | | | | | | | | |
| Comparative | Powder 7 | Powder 2 | 4.9 | 10.1 | 2500.0 | 24.7 | 92.3 | 0.63 | 0.34 |
| example (19) | | | | | | | | | |
| Comparative | Powder 8 | Powder 2 | 4.8 | 10.5 | 2500.0 | 23.8 | 91.1 | 0.61 | 0.34 |
| example (20) | | | | | | | | | |
| Comparative | Powder 9 | Powder 2 | 4.8 | 11.2 | 2500.0 | 22.4 | 86.1 | 0.64 | 0.33 |
| example (21) | | | | | | | | | |
| Comparative | Powder 6 | Powder 4 | 5.5 | 10.4 | 2500.0 | 24.1 | 95.1 | 0.63 | 0.33 |
| example (22) | | | | | | | | | |
| Comparative | Powder 7 | Powder 4 | 4.9 | 10.6 | 2500.0 | 23.6 | 91.8 | 0.60 | 0.35 |
| example (23) | | | | | | | | | |
| Comparative | Powder 8 | Powder 4 | 4.7 | 10.9 | 2500.0 | 23.0 | 90.7 | 0.61 | 0.31 |
| example (24) | | | | | | | | | |
| Comparative | Powder 9 | Powder 4 | 4.7 | 10.9 | 2500.0 | 22.9 | 84.2 | 0.60 | 0.31 |
| example (25) | | | | | | | | | |
| Comparative | Powder 6 | Granule 2 | 5.4 | 9.9 | 2500.0 | 25.2 | 98.5 | 0.63 | 0.33 |
| example (26) | | | | | | | | | |
| Comparative | Granule 7 | Powder 2 | 4.9 | 10.2 | 2500.0 | 24.4 | 95.4 | 0.64 | 0.30 |
| example (27) | | | | | | | | | |
| Comparative | Granule 8 | Powder 4 | 4.7 | 10.6 | 2500.0 | 23.6 | 91.7 | 0.62 | 0.31 |
| example (28) | | | | | | | | | |
| Comparative | Powder 9 | Granule 4 | 4.7 | 11.4 | 2500.0 | 21.9 | 86.1 | 0.61 | 0.34 |
| example (29) | | | | | | | | | |
| Embodiment (6) | Granule 6 | — | 5.2 | 10.7 | 2500.0 | 23.4 | 123.0 | 0.65 | 0.32 |
| Embodiment (7) | Granule 7 | — | 4.7 | 11.0 | 2500.0 | 22.7 | 121.5 | 0.64 | 0.32 |
| Embodiment (8) | Granule 8 | — | 4.6 | 11.3 | 2500.0 | 22.1 | 121.1 | 0.62 | 0.34 |
| Embodiment (9) | Granule 9 | — | 4.5 | 11.7 | 2500.0 | 21.4 | 113.9 | 0.62 | 0.32 |
| Embodiment (10) | Granule 6 | Granule 2 | 5.5 | 9.5 | 2500.0 | 26.3 | 126.4 | 0.65 | 0.31 |
| Embodiment (11) | Granule 7 | Granule 2 | 4.7 | 10.0 | 2500.0 | 25.1 | 123.2 | 0.64 | 0.35 |
| Embodiment (12) | Granule 8 | Granule 2 | 4.6 | 10.1 | 2500.0 | 24.8 | 121.0 | 0.64 | 0.35 |
| Embodiment (13) | Granule 9 | Granule 2 | 4.5 | 10.9 | 2500.0 | 22.9 | 116.6 | 0.60 | 0.32 |
| Embodiment (14) | Granule 6 | Granule 4 | 5.3 | 10.4 | 2500.0 | 24.1 | 125.2 | 0.62 | 0.34 |
| Embodiment (15) | Granule 7 | Granule 4 | 4.6 | 10.1 | 2500.0 | 24.8 | 121.6 | 0.64 | 0.33 |
| Embodiment (16) | Granule 8 | Granule 4 | 4.5 | 10.5 | 2500.0 | 23.9 | 120.2 | 0.62 | 0.30 |
| Embodiment (17) | Granule 9 | Granule 4 | 4.4 | 11.3 | 2500.0 | 22.1 | 118.7 | 0.63 | 0.33 |
| Embodiment (18) | Granule 6 | Granule 2 | 5.4 | 9.9 | 2500.0 | 25.3 | 125.5 | 0.64 | 0.33 |
| Embodiment (19) | Granule 7 | Granule 2 | 4.7 | 9.8 | 2500.0 | 25.4 | 123.3 | 0.64 | 0.33 |
| Embodiment (20) | Granule 8 | Granule 2 | 4.7 | 10.2 | 2500.0 | 24.4 | 121.2 | 0.65 | 0.30 |
| Embodiment (21) | Granule 9 | Granule 2 | 4.6 | 10.9 | 2500.0 | 23.0 | 118.3 | 0.61 | 0.32 |
| Embodiment (22) | Granule 6 | Granule 4 | 5.3 | 10.2 | 2500.0 | 24.6 | 125.1 | 0.62 | 0.33 |
| Embodiment (23) | Granule 7 | Granule 4 | 4.7 | 10.3 | 2500.0 | 24.3 | 122.9 | 0.64 | 0.33 |
| Embodiment (24) | Granule 8 | Granule 4 | 4.5 | 10.5 | 2500.0 | 23.8 | 121.3 | 0.62 | 0.32 |
| Embodiment (25) | Granule 9 | Granule 4 | 4.4 | 10.5 | 2500.0 | 23.9 | 118.2 | 0.62 | 0.33 |

### [Example 26] Green Organic Light Emitting Element (Electron Transport Layer)

The organic electric element was prepared by a typical method using the organic material according to the embodiment of the present invention as the electron transport layer material. First, 4,4′,4″-Tris[2-naphthyl(phenyl)amino]triphenylamine (hereinafter, abbreviated as 2-TNATA) was vacuum-deposited to a thickness of 60 nm on an ITO layer (anode) formed on a glass substrate to form a hole injection layer, and 4,4-bis[N-(1-naphthyl)-N-phenylamino]biphenyl (hereinafter abbreviated as NPD) was vacuum-deposited to a thickness of 60 nm on the hole injection layer to form a hole transport layer. Next, 4,4′-N,N′-dicarbazole-biphenyl (hereinafter, abbreviated as CBP), as the host material, and tris(2-phenylpyridine)-iridium (hereinafter, abbreviated as Ir(ppy)₃), as the dopant material, were doped on the hole transport layer in a weight ratio of 95:5, depositing a 30 nm-thick light emitting layer. Subsequently, (1,1′bisphenyl)-4-oleato)bis(2-methyl-8-quinolineoleato)aluminum (hereinafter, abbreviated as BAlq) was vacuum-deposited, to a thickness of 10 nm, on the light emitting layer to form a hole blocking layer, and the organic material according to the embodiment was vacuum-deposited to a thickness of 40 nm on the hole blocking layer to form an electron transport layer. Thereafter, LiF, which is an alkali metal halide, was deposited to a thickness of 0.2 nm on the electron transport layer to form an electron injection layer, and Al was then deposited to a thickness of 150 nm to form a cathode, thereby preparing the organic electric element.

[Organic Material 10 According to the Embodiment]

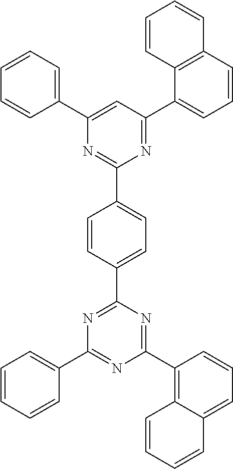 [SMILES: c1ccc(-c2cc(-c3cccc4ccccc34)nc(-c3ccc(-c4nc(-c5ccccc5)nc(-c5cccc6ccccc56)n4)cc3)n2)cc1]

### Embodiment 27 to Embodiment 28

The organic electric element was prepared by the same method as that in embodiment 26 except that organic materials 11 and 12 according to embodiments of the present invention described below were used as the electron transport layer material, instead of organic material 10 according to the embodiment of the present invention.

[Organic Material 11 According to the Embodiment] [Organic Material 12 According to the Embodiment]

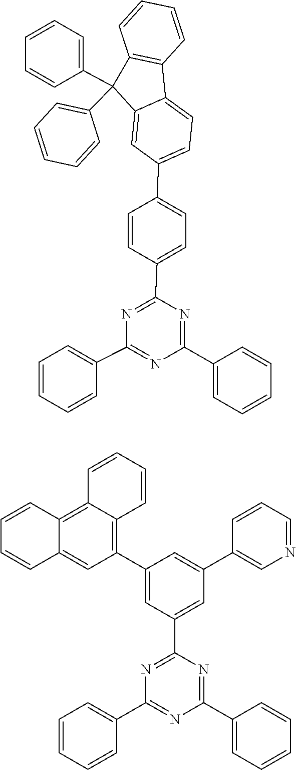 [SMILES: c1ccc(-c2nc(-c3ccccc3)nc(-c3ccc(-c4ccc5c(c4)C(c4ccccc4)(c4ccccc4)c4ccccc4-5)cc3)n2)cc1 ; c1ccc(-c2nc(-c3ccccc3)nc(-c3cc(-c4cccnc4)cc(-c4cc5ccccc5c5ccccc45)c3)n2)cc1]

### Comparative Examples 30 to 32

The organic electric element was prepared by the same method as that in embodiment 26 except that a powdered organic material was used rather than the granular organic material, as the electron transport layer material.

A forward bias DC voltage was applied to the organic electric elements prepared according to embodiments 26 to 28 of the present invention and comparative examples 30 to 32, and the electroluminescence (EL) characteristics were measured with PR-650 of Photo Research Inc., and the T95 lifespan was measured on the measurement result through a lifespan meter manufactured by Mcscience Inc. at the 5000 cd/m² reference luminance. The measurement result is as shown in Table 3 below. In Table 3, the numbers marked after the powders and granules of the organic materials are for distinguishing the types of powders and granules applied to the respective comparative examples and the embodiments.

TABLE 3

| | Organic | Driving | Current | Luminance | Efficiency | | CIE | |
|---|---|---|---|---|---|---|---|---|
| | material | voltage | (mA/cm2) | (cd/m2) | (cd/A) | T(95) | x | y |
| Comparative | Powder 10 | 5.0 | 11.1 | 5000 | 45.2 | 84.7 | 0.34 | 0.61 |
| example (30) | | | | | | | | |
| Comparative | Powder 11 | 5.4 | 11.2 | 5000 | 44.7 | 92.4 | 0.34 | 0.64 |
| example (31) | | | | | | | | |
| Comparative | Powder 12 | 5.2 | 12.2 | 5000 | 41.1 | 89.7 | 0.33 | 0.64 |
| example (32) | | | | | | | | |
| Embodiment (26) | Granule 10 | 4.6 | 10.3 | 5000 | 48.4 | 141.0 | 0.33 | 0.65 |
| Embodiment (27) | Granule 11 | 4.9 | 10.0 | 5000 | 49.8 | 148.9 | 0.33 | 0.65 |
| Embodiment (28) | Granule 12 | 4.8 | 10.4 | 5000 | 47.9 | 137.8 | 0.33 | 0.65 |

From the results for the element, it may be seen that different results for driving, efficiency, and lifespan of the organic electric element are achieved depending on the granular organic material according to the embodiments of the present invention and the powdered organic material according to the comparative examples, i.e., depending on the form of the organic material.

It is identified that the element characteristics differ since the organic materials contain different contents of impurities depending on their form. Referring back to FIG. 7, it could be identified that the content of the impurities contained in the organic material according to the comparative example, which is in the powder form is larger than the content of the impurities contained in the organic material according to the embodiment, which is the granular form, so that the types and amount of the gases generated from the organic material according to the comparative example are larger than the types and amount of the gases generated from the organic material according to the embodiment.

Referring to FIG. 7 and the results for elements according to the embodiments and comparative examples described above, it may be identified that the results of elements are more affected by the applied form of the organic material than by the type of the organic material and the degree of influence of the applied layer. In particular, from comparison between the results of applying the organic material according to the embodiment of the present invention and the organic material according to the comparative example to the light emitting layer, it may be identified that as the content of the granular organic material, as the organic material applied to the light emitting layer, increases, the results of elements are enhanced (driving voltage is reduced).

In other words, it may be identified that as the content of the impurities of the organic material used in forming the light emitting layer reduces, the characteristics of the organic electric element are enhanced.

The above-described embodiments are merely examples, and it will be appreciated by one of ordinary skill in the art various changes may be made thereto without departing from the scope of the present invention. Accordingly, the embodiments set forth herein are provided for illustrative purposes, but not to limit the scope of the present invention, and should be appreciated that the scope of the present invention is not limited by the embodiments. The scope of the present invention should be construed by the following claims, and all technical spirits within equivalents thereof should be interpreted to belong to the scope of the present invention.

### LEGEND OF REFERENCE NUMBERS

200: second material
210: separator
220: first filter
230: second filter
250: organic material
270: residue

### CROSS-REFERENCE TO RELATED APPLICATION

The instant patent application claims priority under 35 U.S.C. 119(a) to Korean Patent Application No. 10-2020-0086374, filed on Jul. 13, 2020, in the Korean Intellectual Property Office, the disclosure of which is herein incorporated by reference in its entirety. The present patent application claims priority to other applications to be filed in other countries, the disclosures of which are also incorporated by reference herein in their entireties.

1-17. (canceled)

18. An organic material for an organic electric element, comprising at least one kind of raw material, wherein a partial area or entire area of a surface of the organic material has a needle shape, and wherein the organic material has a granular form.

19. The organic material of claim 18, wherein the organic material has a form of grains or granules having a particle diameter more than 0.1 mm and equal to or less than 0.5 mm.

20. The organic material of claim 18, wherein the at least one kind of raw material is a material that melts at a 50° C. to 70° C. lower temperature than a temperature at which a weight loss of 0.5% occurs.

21. A method for preparing an organic material for an organic electric element, the method comprising:
a first step of preparing a first material including at least one kind of raw material;
a second step of obtaining a second material by pulverizing the first material; and
a third step of selecting a granular organic material a partial area or entire area of a surface of which has a needle shape from the second material.

22. The method of claim 21, wherein a particle diameter of the organic material is more than 0.1 mm and equal to or less than 0.5 mm.

23. The method of claim 21, wherein the first material includes one kind of raw material, and wherein the first step includes melting the first material.

24. The method of claim 21, wherein the first material includes two or more raw materials.

25. The method of claim 24, wherein the first material includes a first raw material and a second raw material, and wherein the first step includes:
melting each of the first raw material and the second raw material;
solidifying the molten first raw material and the molten second raw material; and
mixing the first raw material and the second raw material.

26. The method of claim 24, wherein the first material includes a third raw material and a fourth raw material, and wherein the first step includes:
mixing the third raw material and the fourth raw material; and
melting and then solidifying the mixed third raw material and fourth raw material.

27. The method of claim 21, wherein the third step includes passing the second material through a separator having a first filter and a second filter disposed on and spaced apart from the first filter and having a particle diameter larger than a particle diameter of the first filter.

28. The method of claim 27, wherein the organic material corresponds to a material remaining on the first filter.

29. The method of claim 27, wherein a particle diameter of the first filter is 0.1 mm or less, and a particle diameter of the second filter is 0.5 mm or less.

30. The method of claim 21, further comprising the step of shaping the organic material.

31. An organic electric element, comprising:
a first electrode;
a second electrode; and
an organic material layer positioned between the first electrode and the second electrode, wherein the organic material layer includes a material corresponding to the organic material of claim 18.

32. The organic electric element of claim 31, wherein the organic material layer includes at least one of a hole injection layer, a hole transport layer, a light emitting auxiliary layer, a light emitting layer, an electron transport auxiliary layer, an electron transport layer, and an electron injection layer, and wherein at least one of the hole injection layer, the hole transport layer, the light emitting auxiliary layer, the light emitting layer, the electron transport auxiliary layer, the electron transport layer, or the electron injection layer included in the organic material layer includes a material including at least one kind of raw material, wherein a partial area or entire area of a surface of the organic material has a needle shape, and wherein the organic material has a granular form.

33. The organic electric element of claim 31, wherein the organic material layer includes a light emitting layer, and wherein the light emitting layer includes a material corresponding to the organic material including at least one kind of raw material, wherein a partial area or entire area of a surface of the organic material has a needle shape, and wherein the organic material has a granular form.

34. The organic electric element of claim 33, wherein a host material of the light emitting layer includes a material corresponding to the organic material.
